(19) United States
(12) Patent Application Publication
(10) Pub. No.: US 2003/0114814 A1
(43) Pub. Date: Jun. 19, 2003

(54) Zoned absorbency with uniform basis weight core material

(76) Inventors: Andrew Thomas Baker, Lawrenceville, GA (US); Dennis Chia-Bin Chen, Alpharetta, GA (US); Douglas Raymond Frederisy JR., Alpharetta, GA (US)

Correspondence Address: Christopher C. Campbell, Esq., HUNTON & WILLIAMS, 1900 K Street NW, Washington, DC, 20006-1109, US

(21) Appl. No.: 10/023,772

(22) Filed: Dec. 17, 2001

(51) Int. Cl. A61F 13/15

(52) U.S. Cl. 604/368; 604/378

(57) ABSTRACT

An absorbent garment having an absorbent core with zones of enhanced absorbency is described. The zones of enhanced absorbency are formed by selective transverse folding of the absorbent core material. Customization of positioning and configuration of the folds facilitates achieving a high distribution index value and accommodating specialized requirements of specific user groups such as male and female users for example.

### BACKGROUND OF THE INVENTION

1. Field of the Invention

The present invention relates generally to an absorbent core for an absorbent garment and more particularly to an absorbent core with zones of enhanced absorbency formed by selective transverse folding of the absorbent core material.

2. Description of Related Art

Disposable absorbent garments such as infant diapers or training pants, adult incontinence products and other such products typically are constructed with a moisture-impervious outer backing sheet, a moisture-pervious body-contacting inner liner sheet, and a moisture-absorbent core sandwiched between the liner and backing sheets.

Much effort has been expended to find cost-effective absorbent cores that display good liquid absorbency and retention. In many applications, it is desirable to form an absorbent article having a zoned absorbency profile where different predetermined regions have different basis weights of fibers per unit area, and, therefore, different absorbencies. Such variation in basis weight across a fibrous article can, for example, enhance the efficiency of the fibrous article in end usage as disposable diapers and sanitary napkins.

For example, U.S. Pat. No. 4,685,915 to Hasse et al. discloses a disposable diaper wherein a central portion of its absorbent core has a higher density and higher basis weight per unit area than longitudinally placed end portions of the absorbent core. The disclosed absorbent cores may comprise a mixture of hydrophilic fibers and discrete particles of a highly absorbent material such as, for example, hydrogel material. The absorbent cores are described as having a central portion that is preferably substantially uniformly dense and of uniform basis weight throughout its extent, and where the ratio of the average density of the central portion to the average density of each of the end portions is about 2 to 1 or greater, and more preferably, 2.5 to 1 or greater.

U.S. Pat. No. 5,849,002 to Carlos et al. discloses a disposable diaper in which the absorbent core material is distributed in such a way that three zones are made inside of it, one of liquid reception, a zone of distribution-storage, and finally an anti-leakage zone. The reception zone is described as being placed where generally the user discharges urine while using the diaper. This zone is described as being less dense and has a lower specific gravity than the distribution-storage zone that fully surrounds it, in such a way that when liquid flows into the reception zone it is immediately absorbed and flows towards the distribution-storage zone, which will distribute the liquid to every zone of the diaper and where it will then remain until the diaper is disposed.

U.S. Pat. No. 5,817,079 to Bergquist et al. discloses absorbent products having discrete areas of dry fibrous materials such as fluid-repellent materials that are precisely placed in various planes within the product so as to provide barriers to bodily fluid leakage from the product. A preferred embodiment is described as having hydrophilic fibers placed around the perimeter of a central absorbent area of an absorbent product to discourage and/or prevent side or end leakage from the product.

U.S. Pat. No. 5,098,423 to Pieniak et al. discloses a disposable diaper that is particularly configured for improved fit and comfort, as well as to provide enhanced absorptive capacity and leakage-resistant characteristics. The disposable diaper is described as having improved fit and comfort that is achieved by providing the diaper with a relatively thin, narrow absorbent panel, with the panel configured to provide desirably high absorbency efficiency for enhanced performance characteristics. The diaper is described as having an absorbent panel that is configured to exhibit sufficient and inherent absorbency to provide an impact zone with an absorbent efficiency index of at least 1.5. This absorbency efficiency index is determined by ascertaining the absorptive characteristics of a diaper's absorbent panel as it relates to the bulk of the panel. The absorbency efficiency index relates to the relationship between the diaper bulk and absorptive capacity. This index is described as being determined by dividing the urine volume at the 90^{th} percentile level in the impact zone (of a mid-size diaper) by the volume of the impact zone absorptive medium. The impact zone is defined as the 6 inches of panel located at the second and third fifths of the panel length, as measured from the front of the diaper.

U.S. Pat. No. 4,997,428 to Linnebur et al. discloses a hygienic disposable article used as a diaper, which possesses an absorbent body enhanced with expandable material. The article is described as having expandable material that is to be applied along the direction of the longitudinal axis of the diaper, in decreasing quantity outward from the crotch area in such a way that the concentration of the expandable material in the crotch area is 8% to 40% of the weight of the absorbent body, while in the area of the waist it is 1% to 7% thereof. The disposable article is described as achieving an optimal distribution of the absorbent components of the diaper in accordance with the distribution pattern of the fluid excreted from the wearer.

Effort has also been expended to find cost effective materials that display good liquid absorbency and retention. Superabsorbent materials in the form of granules, beads, fibers, bits of film, globules, etc., have been favored for such purposes. Such superabsorbent materials are generally polymeric gelling materials (superabsorbent polymers e.g., SAP) that are capable of absorbing and retaining even under moderate pressure large quantities of liquid, such as water and body wastes, relative to their weight.

The superabsorbent material generally is a water-insoluble but water-swellable polymeric substance capable of absorbing water in an amount which is at least ten times the weight of the substance in its dry form. In one type of superabsorbent material, the particles or fibers may be described chemically as having a back bone of natural or synthetic polymers with hydrophilic groups or polymers containing hydrophilic groups being chemically bonded to the back bone or in intimate admixture therewith. Included in this class of materials are such modified polymers as sodium neutralized cross-linked polyacrylates and polysaccharides including, for example, cellulose and starch and regenerated cellulose which are modified to be carboxylated, phosphonoalkylated, sulphoxylated or phosphorylated, causing the SAP to be highly hydrophilic. Such modified polymers may also be cross-linked to reduce their water-solubility.

The ability of a superabsorbent material to absorb liquid is dependent upon the form, position and/or manner in which particles of the superabsorbent are incorporated into the absorbent core. Whenever a particle of the superabsorbent material and absorbent core is wetted, it swells and forms a gel. Gel formation can block liquid transmission into the interior of the absorbent core, a phenomenon called “gel blocking.”

In order for superabsorbent materials to function, the liquid being absorbed in the absorbent structure must be transported to unsaturated superabsorbent material. In other words, the superabsorbent material must be placed in a position to be contacted by liquid. Furthermore, as the superabsorbent material absorbs the liquid it must be allowed to swell. If the superabsorbent material is prevented from swelling, it will cease absorbing liquids.

Additionally, adequate absorbency of liquid by the absorbent core at the point of initial liquid contact and rapid distribution of liquid away from this point is necessary to ensure that the absorbent core has sufficient capacity to absorb subsequently deposited liquids.

Use of an absorbent core formed from a flat sheet with not more than 20% of the sheet is SAP in which the SAP is selectively positioned in two of three central longitudinal trisections is described in U.S. Pat. No. 6,066,775 to Bachar. The Bachar '775 Patent further describes forming the absorbent core by folding the outer longitudinal sections over the central section and folding the front portion over the crotch portion to “lock” the SAP in the desired position. This process requires specialized application of the SAP and folding in two directions.

Other patents such as U.S. Pat. No. 5,681,300 to Ahar et al. discloses folding layers associated with the absorbent core in the longitudinal direction. More particularly the Ahar '300 Patent discloses folding an acquisition layer sheet that improves wicking to the absorbent core in the longitudinal direction in a manner that gives the acquisition layer sheet the appearance of a “z” when viewed in the transverse direction.

Use of an absorbent core formed from at least one and preferably more than one laminate comprising three layers, one of which is a central fibrous layer containing from about 50-90% by weight superabsorbent polymer was described by Chmielewski in U.S. Pat. No. 6,068,620. The Chmielewski '620 Patent is incorporated herein by reference in its entirety in a manner consistent with this disclosure. As described by Chmielewski, further improvement in absorbency may be achieved by using multiple discrete laminates. The Chmielewski '620 Patent also described using laminates C-folded in the machine direction to facilitate positioning of the multiple laminates and/or minimizing side leakage. The absorbent core described by the Chmielewski '620 Patent offers many advantages over prior art including efficient utilization of SAP at concentrations of greater than 50% by weight in the absorbent core.

The documents described above with reference to related are all are expressly incorporated by reference herein in their entirety.

### SUMMARY OF INVENTION

Because the primary area of insult is not uniformly distributed over the entire core, it would be desirable to have an absorbent core with high concentrations of SAP and selectively positioned zones of increased absorbency which enhance distribution efficiency and decreases the bulk of multiple discrete layers of laminate. Further, a need exists that such a product have sufficient machine direction strength to survive folding forces and sufficient flexibility to avoid formation of hard edges. It is therefore a feature of various embodiments of the invention to provide an absorbent core, absorbent article, and method of making the same, whereby the absorbent core has selectively positioned zones of varying absorbency that increases the efficiency thereof.

These and other features of embodiments of the invention are achieved by providing an absorbent garment having a zoned absorbency core formed from uniform basis weight core material, and having a good distribution efficiency. The absorbent article comprises a liquid permeable top sheet; a liquid impermeable back sheet associated with the top sheet; and an absorbent core disposed between the top sheet and the back sheet. The absorbent core comprises an absorbent laminate, the absorbent laminate having an upper layer near the top sheet; a lower layer near the back sheet; and an absorbent layer disposed between the upper and lower layers. Although not required in the invention, it is preferred that the side edges of the absorbent layer are substantially uncovered by the upper and lower layers. The upper and lower layers of the laminate preferably comprise a material selected from the group consisting essentially of tissue, air laid fluff pulp and synthetic non-woven. The absorbent layer comprises from about 10-90% SAP by weight. The absorbent laminate has a first end; a second end; at least one transverse fold; and a layer region of two or more layers positioned between the first end and the second end. The layer region has a plurality of layer edges at least one of the plurality of layer edges coinciding with the at least one transverse fold and at least one of the plurality of layer edges is positioned at a distance intermediate the first end and the second end.

In accordance with an additional feature of an embodiment of the invention, there is provided an absorbent core for absorbent garments . The absorbent core comprises a roll good core portion. The roll good core portion has a first end; a second end; at least one transverse fold, and a layer region of two or more layers positioned between the first end and the second end. The layers have a plurality of layer edges, at least one of the plurality of layer edges coincides with the at least one transverse fold and at least one of the plurality of layer edges is positioned at a position intermediate the first end and the second end.

Another feature of an embodiment of the invention includes a method of manufacturing an absorbent core for absorbent garments. The method comprises providing a roll good core portion, having a first end and a second end to a folding station including a rotating drum having grasping projections, the first end being the leading end and the second end being the trailing end;

drawing the leading end into a chamber disposed adjacent the folding station;

engaging the roll good core portion with the grasping projections of the rotating drum.

advancing the trailing portions of the roll good core portion along the rotating drum and past the chamber;

creating a fold in the roll good core portion as the trailing portions of the roll good core portion is advanced past the chamber; and

releasing the leading end from the chamber prior to the trailing end advancing past the chamber on the rotating drum and releasing the roll good core portion from the grasping projections.

In accordance with another feature of an embodiment of the invention, the above method is repeated at least once to provide an absorbent core having two folds. Another feature of an embodiment of the invention includes a method of forming an absorbent garment that includes:

supplying a top sheet material;

supplying a back sheet material;

preparing an absorbent core in accordance with the method described above; and

disposing the absorbent core between the top sheet material and the back sheet material.

Another feature of an embodiment of the invention includes a method of making an absorbent core. The method comprises providing a roll good core web, having a first web portion, a layer segment and a second web portion to a folding station;

advancing the roll good core web to a folding station having a first moving belt, a second moving belt and a pressor wherein the first web portion of the roll good core web is drawn along the first moving belt and the layer segment portion is drawn along the second moving belt;

creating at least one loop in the roll good core web as the roll good core is drawn along by the first and the second moving belts;

pressing at least one loop into at least one fold with the pressor;

feeding the second portion of the roll good core web along one of the first and second moving belts; and

cutting a length of roll good core web wherein the length of roll good core web comprises at least one fold and a portion of the one of the first and second web portions.

Another feature of an embodiment of the invention includes a method of forming an absorbed garment that includes:

supplying a top sheet material;

supplying a back sheet material;

preparing an absorbent core from a roll good core web in accordance with the method described above; and

disposing the absorbent core between the top sheet material and back sheet material.

### BRIEF DESCRIPTION OF THE DRAWINGS

The invention may be understood more readily by reviewing the drawings, in which:

FIG. 1 depicts a partially cut away view of an exemplary embodiment of an absorbent garment;

FIG. 2 illustrates a side view of a folded absorbent core having a single fold positioned in the cross direction;

FIG. 3 illustrates a side view of a folded absorbent core having two folds in the cross direction and providing enhanced absorbency potential at a position inward from the absorbent core ends;

FIG. 4 depicts a folded absorbent core with two folds positioned such that three zones of differing absorbency potential are formed;

FIG. 5 shows a diagram showing the position of points i,j on a core in an absorbent article and the positioning of a grid used to measure DI with respect to an absorbent garment and the core thereof.

FIG. 6 is a graph illustrating insult points on diapers in a gender-based study;

FIG. 7 is a graph illustrating insult points on diapers in a gender-based study;

FIG. 8 is a graph illustrating variation in insult point over time in a gender-based study; and

FIG. 9 is a schematic illustration of one method for forming a single fold absorbent core.

FIG. 10 is a schematic illustration of one method for forming an absorbent core with multiple folds.

### DETAILED DESCRIPTION OF PREFERRED EMBODIMENTS

One advantage of an embodiment of the invention is that by selectively folding the absorbent core material, zones of enhanced absorbing capability may be strategically positioned in absorbent garments. This strategic positioning may permit enhancement of distribution efficiency as well as reduction of the overall bulk of core material needed to achieve the desired absorbency or both. This leads to two important advantages, namely, a less bulky more comfortable garment and reduced cost of materials. Yet another advantage of the invention is that the folding proposed herein can be accomplished cost effectively and quickly in the manufacturing environment.

As used herein, the term “absorbent garment” refers to garments that absorb and contain exudates, and more specifically, refers to garments which are placed against or in proximity to the body of the wearer to absorb and contain the various exudates discharged from the body. A nonexhaustive list of examples of absorbent garments includes diapers, diaper covers, disposable diapers, training pants, feminine hygiene products and adult incontinence products. The term “disposable absorbent garment” refers to absorbent garments that are intended to be discarded or partially discarded after a single use (i.e., they are not intended to be laundered or otherwise restored or reused). The term “unitary disposable absorbent garment” refers to a disposable absorbent garment that is essentially a single structure (i.e., it does not require separate manipulative parts such as a diaper cover and insert). As used herein, the term “diaper” refers to an absorbent garment generally worn by infants and incontinent persons about the lower torso.

The claims are intended to cover all of the foregoing classes of absorbent garments, without limitation, whether disposable, unitary or otherwise. These classifications are used interchangeably throughout the specification, but are not intended to limit the claimed invention. The invention will be understood to encompass, without limitation, all classes of absorbent garments, including those described above. Preferably, the absorbent core is thin in order to improve the comfort and appearance of a garment. The importance of thin, comfortable garments is disclosed, for example, in U.S. Pat. No. 5,098,423 to Pieniak et al., which is herein incorporated by reference.

Throughout this description, the expressions “upper layer,” “lower layer,” “above” and “below,” which refer to the various components included in the absorbent core units of the invention (including the layers surrounding the absorbent core units) are used merely to describe the spatial relationship between the respective components. The upper layer or component “above” the other component need not always remain vertically above the core or component, and the lower layer or component “below” the other component need not always remain vertically below the core or component. Indeed, embodiments of the invention include various configurations whereby the core is folded in such a manner that the upper layer ultimately becomes the vertically highest and vertically lowest layer at the same time. Other configurations are contemplated within the context of the present invention.

The term “component” can refer, but is not limited, to designated selected regions, such as edges, corners, sides or the like; structural members, such as elastic strips, absorbent pads, stretchable layers or panels, layers of material, or the like; or a graphic. The term “graphic” can refer, but is not limited, to any design, pattern, indicia or the like.

Throughout this description, the term “disposed” and the expressions “disposed on,” “disposing on,” “disposed in,” “disposed between” and variations thereof (e.g., a description of the article being “disposed” is interposed between the words “disposed” and “on”) are intended to mean that one element can be integral with another element, or that one element can be a separate structure bonded to or placed with or placed near another element. Thus, a component that is “disposed on” an element of the absorbent garment can be formed or applied directly or indirectly to a surface of the element, formed or applied between layers of a multiple layer element, formed or applied to a substrate that is placed with or near the element, formed or applied within a layer of the element or another substrate, or other variations or combinations thereof.

Throughout this description, the terms “topsheet” and “backsheet” denote the relationship of these materials or layers with respect to the absorbent core. It is understood that additional layers may be present between the absorbent core and the topsheet and backsheet, and that additional layers and other materials may be present on the side opposite the absorbent core from either the topsheet or the backsheet.

Throughout this description, the expression “tow fibers” relates in general to any continuous fiber. Tow fibers typically are used in the manufacture of staple fibers, and preferably are comprised of synthetic thermoplastic polymers. Usually, numerous filaments are produced by melt extrusion of the molten polymer through a multi-orifice spinneret during manufacture of staple fibers from synthetic thermoplastic polymers in order that reasonably high productivity may be achieved. The groups of filaments from a plurality of spinnerets typically are combined into a tow which is then subjected to a drawing operation to impart the desired physical properties to the filaments comprising the tow.

Absorbent garments and diapers may have a number of different constructions. In each of these constructions it is generally the case that an absorbent core is disposed between a liquid pervious body-facing topsheet, and a liquid impervious, exterior facing backsheet. In some cases, one or both the topsheet and the backsheet may be shaped to form a pant-like garment. In other cases, the topsheet, backsheet and absorbent core may be formed using a discreet assembly that is placed on a main chassis and the chassis is made to form a pant-like garment. In the case of diapers, a caregiver usually wraps the diaper around the wearer's waist and joins the side seams manually by attaching one or more adhesive or mechanical tabs, thereby making the pant-like structure. In the case of training pant-type garments and most adult incontinent products, the garment is provided fully formed with factory made side seams and the garment is donned by pulling it up the wearer's leg. For clarity, the present invention is described herein, only with reference to the diaper type garment. Although the invention may be used with other constructions including for example, the training pant-type garments, adult and incontinent products or feminine hygiene products.

The present invention relates to an absorbent article comprising a core that can be designed to have selectively positioned zones of absorbency by folding the absorbent core in a variety of configurations. the absorbent core preferably is comprised of a laminate including an upper layer, a lower layer, and a central layer that contains a superabsorbent polymer (SAP). The central layer preferably also includes a fibrous material that may be selected from the group consisting essentially of tow fibers, cellulose acetate fibers, rayon fiber, LYOCELL fibers, polyacrylonitrite fibers, cotton fibers and cotton linter fibers or from the groups consisting essentially of surface modified (hydrophilic) polyester fibers and surface modified polyolefin/polyester bicomponent fibers.

In some preferred embodiments the absorbent layer may comprise about 20-90% SAP by weight. The absorbent layer may further comprise particulate additives. In other preferred embodiments the particle additives comprise insoluble hydrophilic polymers with particle diameters of 100 μm or less.

In one embodiment, at least one transverse fold is comprised of two folds. In another preferred embodiment the plurality of layer edges of the layer region are at a position intermediate the first and second ends. In yet another preferred embodiment the layer region is positioned for either a female user or a male user. The absorbent article preferably is diaper, and more preferably, an extended use diaper.

The invention also relates to an absorbent core that includes a roll good core material having an upper layer, a lower layer, and a center layer containing SAP. The center layer may also contain a fibrous material selected from the group consisting essentially of tow fibers, cellulose acetate fibers, rayon fibers, LYOCELL fibers, polyacrylonitrile fibers, surface modifier (hydrophilic) polyester fibers, surface-modified polyolefin/polyester bicomponent fibers, surface-modified polyester/polyester bicomponent fibers and cotton fibers.

In one preferred embodiment the roll good core portion may further comprise about 10-90% SAP by weight. In another preferred embodiment the roll good core portion may comprise about 20-90% by weight SAP. The roll good core portion may further comprise particulate additives.

In some preferred embodiments of the absorbent core, the plurality of layer edges of the layer region are positioned intermediate the first and second ends of the absorbent core. In a preferred embodiment of the absorbent core, the at least one transverse fold includes two folds. In another preferred embodiment of the absorbent core the layer region is positioned for either a female user or a male user.

The invention also relates to a method of making an absorbent core for an absorbent garment. In one embodiment the method of making an absorbed core comprises providing a roll good core portion having a first end (e.g., leading end) and a second end to a folding station including a rotating drum having grasping projections. The first end is drawn into a chamber disposed adjacent the folding station and the grasping projections engage the roll good core portion. The trailing portions of the roll good core portion are advanced along the rotating drum and past the chamber. A fold is created in the roll good core as the trailing portions of the roll good core portion advance past the chamber. The leading end is released from the chamber prior to the second end (e.g., the trailing end) advancing past the chamber on the rotating drum and the roll good core is released from the grasping projections.

In another embodiment the method of making an absorbent core comprises providing a roll good core web, having a first portion, a layer segment and a second portion. The roll good core web is advanced to a folding station having a first moving belt, a second moving belt and a pressor. The first portion of the roll good core web is drawn along the first moving best and the layer segment portion is drawn along the second moving belt. At least one loop is created in the roll good core web as the roll good core is drawn along by the first and second moving belts. At least one loop is pressed into at least one fold by the pressor. The second portion of the roll good core web is fed along one of the first and second moving belts and a length of roll good core web comprising a portion of at least one of the first and second web portion and at least one fold is cut to form an individual absorbent core. In some embodiments the pressor is a nip drum. In a preferred embodiment, the belts are vacuum belts.

The invention now will be described with reference to the attached drawings illustrating preferred embodiments of the invention. For clarity, features that appear in more than one Figure have the same reference number in each Figure.

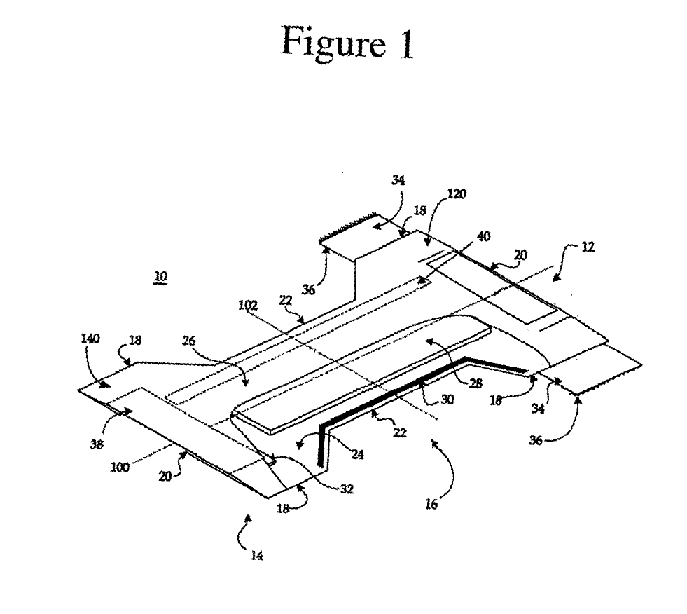

FIG. 1 is a partially cut away depiction of an exemplary embodiment of an absorbent garment 10 (preferably a disposable absorbent garment) of the present invention. The embodiment shown in FIG. 1 is an infant's diaper, however, this depiction is not intended to limit the invention, and those skilled in the art appreciate that the invention covers other types of absorbent articles. For simplicity, however, the invention will be described with reference to an infant's diaper. The garment 10 of FIG. 1 is depicted in a generally flattened position, with the body-facing side facing down, and with the various elastic components depicted in their extended condition with the effects of the elastics removed for clarity (when relaxed, the elastics typically cause the surrounding material to gather or “shirr”). In the flattened position, the garment 10 may have a generally hourglass shaped structure, but it may also have any other shape suitable for the given application, such as a rectangular shape, a trapezoidal shape, a “T” shape, and the like.

As used herein, the longitudinal axis 100 of the garment is the dimension of the garment corresponding to the front-to-rear dimension of the user, and the lateral axis 102 of the garment is the dimension corresponding to the side-to-side dimension of the user.

In use, the invention comprises a pant-like garment 10 having a waist-encircling region and a crotch region. The waist-encircling region may comprise a first waist region 12, disposed adjacent to, for example, the back waist region of a wearer's body, and a second waist region 14, disposed adjacent to, for example, the front waist region of a wearer's body. The first and second waist regions 12, 14, may correspond to the front and back of the wearer's body, respectively, depending on whether garment 10 is attached in front of or behind the subject wearer. The first and second waist regions are joined together at or near their lateral edges 18, causing the longitudinally distal edges 20 of the garment 10 to form the perimeter of a waist opening. A crotch region 16 extends between the first and second waist regions 12, 14, and the crotch edges 22 form the perimeter of a pair of leg openings, when the garment 10 is placed on a subject wearer.

The garment 10 preferably comprises a topsheet 24, and a backsheet 26, which may be substantially coterminous with the topsheet 24. When the garment 10 is being worn, the topsheet 24 faces the wearer's body, and the backsheet 26 faces away from the wearer. An absorbent core 28 preferably is disposed between at least a portion of the topsheet 24 the backsheet 26.

An embodiment of the present invention may further comprise various additional features. One or more pairs of elastic gathers 30 may extend adjacent the crotch edges 22. The garment 10 may also comprise one or more waste containment systems, such as inboard standing leg gathers 40, which preferably extend from the second waist region 14 to the first waist region 12 along opposite sides of longitudinal center line 100 (only one standing leg gather system 40 is shown in FIG. 1 for purposes of clarity). One or both of the first and second waist regions 12, 14 may also be equipped with strips of elastic waist foam 32 or other elastically extensible material, which help contract the garment around the wearer's waist, providing improved fit and leakage prevention.

The absorbent garment 10 also preferably includes fastening elements to enable attachment of the first waist region 12 to second waist region 14. Fastening elements preferably include a pair of tabs 34 that extend laterally away from opposite lateral edges 18 of the first waist region 12 of the garment 10. The tabs 34 may comprise an elastically extensible material (not shown), and may be designed to stretch around a wearer's waist to provide improved fit, comfort, and leakage protection. Such elasticized tabs 34 may be used in conjunction with, or in lieu of, waist foam 32, or other elastically extensible materials 32.

At least one fastening mechanism 36 (collectively referred to as “fastener 36”) is attached to each tab 34 for attaching the tab to the second waist region 14, thereby providing the garment 10 with a pant-like shape, and enabling garment 10 to be fixed or otherwise fitted on the wearer. The fasteners 36 may attach to one or more target devices 38 located in the second waist region 14.

Although not shown in the drawings, the absorbent garment 10 may also include grips attached along one of its edges proximal to each tab 34 to enable a caregiver to pull the grips, and not on the ends of the tabs 34, around the wearer and over the target devices 38 to thereby secure the fasteners 36 to the one or more target devices 38.

The various parts of the garment 10 can be attached to one another or associated with one another to form a structure that preferably maintains its shape during the useful life of the garment 10. As used herein, the terms “attached,” “joined,” “associated,” and similar terms encompass configurations whereby a first part is directly joined to a second part by affixing the first part directly to the second part, by indirectly joining the first part to the second part through intermediate members, and by fixing the relative positions of various parts by capturing parts between other parts. Those skilled in the art will appreciate that various methods or combinations of methods may be used to securely join the respective parts of the garment 10 to one another.

The topsheet 24 and backsheet 26 may be constructed from a wide variety of materials known in the art. Due to the wide variety of backing and liner sheet construction and materials currently available, the invention is not intended to be limited to any specific materials or constructions of these components. The topsheet 24 and backsheet can be shaped and sized according to the requirements of each of the various types of absorbent garment, or to accommodate various user sizes. In an embodiment of the invention in which the garment 10 is a diaper or an adult incontinence brief, the combination of topsheet 24 and backsheet 26, may have an hourglass shape, as seen in FIG. 1, or may have a rectangular, trapezoidal, “T” shape, or other shape.

The back sheet 26 preferably is made from any suitable pliable liquid-impervious material known in the art. Typical back sheet materials include films of polyethylene, polypropylene, polyester, nylon, and polyvinyl chloride and blends of these materials. For example, the back sheet can be made of a polyethylene film having a thickness in the range of 0.02-0.04 mm. The backsheet 26 may be pigmented with, for example, titanium dioxide, to provide the garment 10 with a pleasing color or to render the backsheet 26 opaque enough that exudates being contained by the garment 10 are not visible from outside the garment. In addition, the backsheet 26 may be formed in such a manner that it is opaque, for example, by using various inert components in the polymeric film and then biaxially stretching the film. Other backsheet materials will be readily apparent to those skilled in the art. The backsheet 26 preferably has sufficient liquid imperviousness to prevent any leakage of fluids. The required level of liquid imperviousness may vary between different locations on the garment 10.

The backsheet 26 may further comprise separate regions having different properties. In a preferred embodiment, portions of the backsheet 26 are air-permeable to improve the breathability, and therefore comfort, of the garment 10. The different regions may be formed by making the backsheet 26 a composite of different sheet materials, chemical treatment, heat treatment, or other processes or methods known in the art. Some regions of the backsheet 26 may be fluid pervious. In one embodiment of the invention, the backsheet 26 is fluid impervious in the crotch 16, but is fluid pervious in portions of the first and second waist regions 12, 14. The backsheet 26 may also be made from a laminate of overlaid sheets of material.

The moisture-pervious top sheet 24 can be comprised of any suitable relatively liquid-pervious material known in the art that permits passage of liquid there through. Non-woven liner sheet materials are exemplary because such materials readily allow the passage of liquids to the underlying absorbent core 28. Examples of suitable liner sheet materials include non-woven spunbond or carded webs of polypropylene, polyethylene, nylon, polyester and blends of these materials.

The backsheet 26 may be covered with a fibrous, nonwoven fabric such as is disclosed, for example, in U.S. Pat. No. 4,646,362 issued to Heran et al., the disclosure of which is hereby incorporated by reference in its entirety and in a manner consistent with this disclosure. Materials for such a fibrous outer liner include a spun-bonded nonwoven web of synthetic fibers such as polypropylene, polyethylene or polyester fibers; a nonwoven web of cellulosic fibers, textile fibers such as rayon fibers, cotton and the like, or a blend of cellulosic and textile fibers; a spun-bonded nonwoven web of synthetic fibers such as polypropylene; polyethylene or polyester fibers mixed with cellulosic, pulp fibers, or textile fibers; or melt blown thermoplastic fibers, such as macro fibers or micro fibers of polypropylene, polyethylene, polyester or other thermoplastic materials or mixtures of such thermoplastic macro fibers or micro fibers with cellulosic, pulp or textile fibers. Alternatively, the backsheet 26 may comprise three panels wherein a central poly backsheet panel is positioned closest to absorbent core 28 while outboard non-woven breathable side backsheet panels are attached to the side edges of the central poly backsheet panel. Alternatively, the backsheet 26 may be formed from microporous poly coverstock for added breathability.

The top sheet 24 may be formed of three separate portions or panels. Those skilled in the art will recognize, however, that top sheet 24 need not be made of three separate panels, and that it may be comprised of one unitary item. A first top sheet panel (not shown) may comprise a central top sheet panel formed from preferably a liquid-pervious material that is either hydrophobic or hydrophilic. The central top sheet panel may be made from any number of materials, including synthetic fibers (e.g., polypropylene or polyester fibers), natural fibers (e.g., wood or cellulose), apertured plastic films, reticulated foams and porous foams to name a few. One preferred material for a central top sheet panel is a cover stock of single ply non-woven material which may be made of carded fibers, either adhesively or thermally bonded, perforated plastic film, spunbonded fibers, or water entangled fibers, which generally weigh from 0.3-0.7 oz./sq. yd. and have appropriate and effective machine direction and cross-machine direction strength suitable for use as a baby diaper cover stock material. The central top sheet panel preferably extends from substantially the second waist region 14 to the first waist region 12, or a portion thereof.

A second and third top sheet panels (e.g., outer top sheet panels, not shown), in this alternative embodiment may be positioned laterally outside of the central top sheet panel. The outer top sheet panels preferably are substantially liquid-impervious and hydrophobic, preferably at least in the crotch area. The outer edges of the outer top sheet panels may substantially follow the corresponding outer perimeter of the back sheet 26. The material for the outer top sheet portions or panels is preferably polypropylene and can be woven, non-woven, spunbonded, carded or the like, depending on the application.

At the point of connection between the outer topsheet panels and the central topsheet panel, inner edges of the outer topsheet portions or panels may extend upwardly to form waste containment flaps 40. The waste containment flaps 40 preferably are formed of the same material as the outer topsheet portions or panels, as in the embodiment shown. The waste containment flaps 40 may be treated with a suitable surfactant to modify their hydrophobicity/hydrophilicity as desired, and they may be treated with skin wellness ingredients to reduce skin irritation. Alternatively, the waste containment flaps 40 may be formed as separate elements and then attached to the body side liner.

The waste containment flaps 40 preferably include a portion that folds over onto itself to form a small enclosure. At least one, and depending on the size of the enclosure sometimes more than one, elastic member may be secured in the enclosure in a stretched condition. When the flap elastic attempts to assume the relaxed, unstretched condition, the waste containment flaps 40 rise above the surface of the central topsheet portion or panel.

The topsheet 24 may be made of any suitable relatively liquid-pervious material currently known in the art or later discovered that permits passage of a liquid there through. Examples of suitable topsheet materials include nonwoven spun-bonded or carded webs of polypropylene, polyethylene, nylon, polyester and blends of these materials, perforated, apertured, or reticulated films, and the like. Nonwoven materials are exemplary because such materials readily allow the passage of liquids to the underlying absorbent core 28. The topsheet 24 preferably comprises a single-ply nonwoven material that may be made of carded fibers, either adhesively or thermally bonded, spunbonded fibers, or water entangled fibers, which generally weigh from 0.3-0.7 oz./sq. yd. and have appropriate and effective machine direction (longitudinal) and cross-machine (lateral) direction strength suitable for use as a topsheet material for the given application. The present invention is not intended to be limited to any particular material for the topsheet 24, and other topsheet materials will be readily apparent to those skilled in the art.

The topsheet 24 may further comprise several regions having different properties. In one embodiment of the present invention, the laterally distal portions of the topsheet 24, especially those used to make the outer top sheet panels preferably are substantially fluid impervious and hydrophobic, while the remainder of the topsheet 24 (e.g., central top sheet panel) is hydrophilic and fluid pervious. Different topsheet properties, such as fluid perviousness and hydrophobicity, may be imparted upon the topsheet 24 by treating the topsheet 24 with adhesives, surfactants, or other chemicals, using a composite of different materials, or by other means. The topsheet 24 may also be made from a laminate of overlaid sheets of material. The topsheet 24 also may be treated in specific areas like the crotch region, with skin wellness ingredients such as aloe, vitamin E, and the like.

As noted elsewhere herein, the topsheet 24 and backsheet 26 may be substantially coterminous, or they may have different shapes and sizes. The particular design of the topsheet 24 and backsheet 26 may be dictated by manufacturing considerations, cost considerations, and performance considerations. Preferably, the topsheet 24 is large enough to completely cover the absorbent core 28, and the backsheet 26 is large enough to prevent leakage from the garment 10. The design of topsheet 24 and backsheet 26 is known in the art, and a skilled artisan will be able to produce an appropriate topsheet 24 and an appropriate backsheet 26 without undue experimentation.

The topsheet 24 and the backsheet 26 may be associated with one another using a variety of methods known in the art. For example, they may be thermally, ultrasonically, or chemically bonded to one another. They also may be joined using lines of hot melt adhesive or mechanical fasteners, such as thread, clips, or staples. In one embodiment, a hydrophilic adhesive, such as Cycloflex as sold by National Starch, a corporation headquartered in Bridgewater, N.J., is used to join the topsheet 24 to the backsheet 26. The particular joining method may be dictated by the types of materials selected for the topsheet 24 and backsheet 26.

As mentioned above, the absorbent garment preferably is provided with leg elastics 30 extending through crotch region 16, adjacent crotch edge 22. The absorbent garment of the invention also preferably is provided with waist elastic material 32 optionally in the first and second waist regions, 12, 14, respectively, to enable and assist in stretching around the wearer. The waist elastics 32 may be similar structures or different to impart similar or different elastic characteristics to the first and second waist regions 12, 14 of the garment. In general, the waist elastics may preferably comprise foam strips positioned at the first and second waist regions 12, 14, respectively. Such foam strips preferably are about ½ to about 1½ inches in the dimension parallel to longitudinal axis 100 and about 3-6 inches in the dimension parallel to the lateral axis 102. The foam strips preferably are positioned between the top sheet 24 and the back sheet 26. Alternatively, a plurality of elastic strands may be employed as waist elastics rather than foam strips. The foam strips preferably are comprised of polyurethane, but can be any other suitable material that decreases waist band roll over, reduces leakage over the waist ends of the absorbent garment, and generally improve comfort and fit. The first and optional second waist foam strips 32 preferably are stretched 50-150%, preferably 100% more than their unstretched dimension before being adhesively secured between the back sheet 26 and top sheet 24.

Each edge 22 that forms the leg openings preferably is provided with an adjacent leg elastic containment system 30. In the preferred embodiment, three strands of elastic threads are positioned to extend adjacent to leg openings between the topsheet 24 and the back sheet 26. Any suitable elastomeric material exhibiting at least an elongation (defined herein as (L_{S}−L_{R})/L_{R} where L_{S} is the stretch length of an elastic element and L_{R} is retracted length, multiplied by 100 to obtain percent elongation) in the range of 5%-350%, preferably in the range of 200%-300%, can be employed for the leg elastics 30. The leg elastics 30 may be attached to the absorbent article 10 in any of several ways which are known in the art. For example, the leg elastics 30 may be ultrasonically bonded, heat/pressure sealed using a variety of bonding patterns, or glued to the garment 10. Various commercially available materials can be used for the leg elastics 30, such as natural rubber, butyl rubber or other synthetic rubber, urethane, elastomeric materials such as LYCRA (DuPont), GLOSPAN (Globe) or SYSTEM 7000 (Fulflex).

The fastening elements, preferably a fastening system 34 (e.g., tab 34) of the preferred embodiment, is attached to the first waist region 12, and it preferably comprises a tape tab or mechanical fasteners 36. However, any fastening mechanism known in the art will be acceptable. Moreover, the fastening system 34 may include a reinforcement patch below the front waist portion so that the diaper may be checked for soiling without compromising the ability to reuse the fastener. A landing zone or attachment area 38 preferably is provided in the second waist region 14 to permit attachment of the first waist region 12 to the second waist region 14. Alternatively, other absorbent article fastening systems are also possible, including tapes, adhesives, safety pins, buttons, and snaps.

As stated previously, the invention has been described in connection with a diaper. The invention, however, is not intended to be limited to application only in diapers. Specifically, the absorbent cores of the preferred embodiments may be readily adapted for use in other absorbent garments besides diapers, including, but not limited to, training pants, feminine hygiene products and adult incontinence products.

The underlying structure beneath the topsheet 24 may include, depending on the diaper construction, various combinations of elements, but in each embodiment, it is contemplated that the absorbent garment will preferably include an absorbent core 28. For example, additional layers may be disposed between the topsheet 24 and absorbent core 28, and/or other additional layers may be disposed between these layers, or between absorbent core 28 and backsheet 26. The additional layer(s) may include a fluid transfer layer, a fluid handling layer, a storage layer, a wicking layer, a fluid distribution layer, and any other layer(s) known to those having ordinary skill in the art.

Although the absorbent core 28 depicted in FIG. 1 has a substantially rectangular cross-sectional and plan view shape, other shapes may be used, such as a “T” shape or an hourglass shape. The shape of the absorbent core 28 may be selected to provide the greatest absorbency with a reduced amount of material. The absorbent core may be associated with the topsheet 24, backsheet 26, or any other suitable part of the garment 10 by any method known in the art, in order to fix the absorbent core 28 in place. In addition to the respective layers in the absorbent core 28, as will be described in greater detail hereinafter, the overall absorbent core 28 may be enclosed within a tissue wrapping, as disclosed in U.S. Pat. No. 6,068,620, the disclosure of which is incorporated by reference herein in its entirety. Skilled artisans are capable of designing and wrapping a suitable absorbent core 28 of the invention, using the guidelines provided herein.

Any suitable absorbent material may be used for absorbent core 28. Absorbent cores containing a mixture of fibrous material and superabsorbent polymers (SAP) are well known in the art and described, for example, in U.S. Pat. Nos. 5,281,207, and 6,068,620 to Chmielewski, and U.S. Pat. No. 5,863,288, to Baker, the disclosures of each of which are herein incorporated by reference in their entirety and in a manner consistent with this disclosure. The fibrous material can be any fibrous material capable of absorbing fluids, and capable of retaining SAP particles within its matrix. Preferred fibrous materials may be selected from tow fibers, cellulose acetate fibers, rayon fibers, Courtauld's LYOCELL fibers, polyacrylonitrile fibers, surface-modified (hydrophilic) polyester fibers, surface-modified polyolefin/polyester bicomponent fibers, surface-modified polyester/polyester bicomponent fibers, cotton fibers, or blends thereof. In addition, rayon, Courtauld's LYOCELL, polyacrylonitrile, cotton fibers and cotton linters are alternatively preferred. The remaining fibers, surface-modified polyolefin/polyester bicomponent fibers, and surface-modified polyester/polyester bicomponent fibers are also believed to be effective fibrous materials for use in the invention.

It is preferred that the fibrous material include at least a tow fiber. The tow fiber can be any continuous or discontinuous thermoplastic filament tow fiber that is capable of being opened and used in combination with SAP in an absorbent core. Preferably, cellulose ester tow is used as the fibrous material. Non-limiting examples of suitable cellulose esters include cellulose acetate, cellulose propionate, cellulose butyrate, cellulose caproate, cellulose caprylate, cellulose stearate, highly acetylated derivatives thereof such as cellulose diacetate, cellulose triacetate and cellulose tricaproate, and mixtures thereof such as cellulose acetate butyrate. A suitable cellulose ester will include the ability to absorb moisture, preferably is biodegradable, and is influenced not only by the substituent groups but also by the degree of substitution. The relationship between substituent groups, degree of substitution and biodegradability is discussed in W. G. Glasser et al, BIOTECHNOLOGY PROGRESS, vol. 10, pp. 214-219 (1994), the disclosure of which is incorporated herein by reference in its entirety.

Continuous filament tow useful in the present invention is beneficially moisture-absorbent and biodegradable. Accordingly, cellulose acetate tow is typically preferred for use in the invention. Typically, the denier per fiber (dpf) of the tow fiber will be in the range of about 1 to 9, preferably about 3 to 6. For the same weight product, filaments of lower dpf may provide increased surface area and increased moisture absorption. Total denier may vary within the range of about 20,000 to 60,000, depending upon the process used.

It is particularly preferred in the invention to use tow having crimped filaments. Tow materials having crimped filaments are typically easier to open. Separation of filaments resulting from bloom advantageously results in increased available filament surface area for superabsorbent material immobilization and increased moisture absorption. Gel blocking also may be reduced by using crimped tow in the central fibrous containing layer of the absorbent core 28. As therefore may be understood, more crimp is typically better, with in excess of about 20 crimps per inch being usually preferred. Continuous filament, cellulose ester tow having crimped filaments with about 25 to 40 crimps per inch, is commercially available from Hoechst Celanese Corporation, Charlotte, N.C.

Any superabsorbent polymer (SAP) now known or later discovered may be used in absorbent core 28, so long as it is capable of absorbing liquids. Useful SAP materials are those that generally are water-insoluble but water-swellable polymeric substance capable of absorbing water in an amount that is at least ten times the weight of the substance in its dry form. In one type of SAP, the particles or fibers may be described chemically as having a back bone of natural or synthetic polymers with hydrophilic groups or polymers containing hydrophilic groups being chemically bonded to the back bone or in intimate admixture therewith. Included in this class of materials are such modified polymers as sodium neutralized cross-linked polyacrylates and polysaccharides including, for example, cellulose and starch and regenerated cellulose which are modified to be carboxylated, phosphonoalkylated, sulphoxylated or phosphorylated, causing the SAP to be highly hydrophilic. Such modified polymers may also be cross-linked to reduce their water-solubility.

Examples of suitable SAP are water swellable polymers of water soluble acrylic or vinyl monomers crosslinked with a polyfunctional reactant. Also included are starch modified polyacrylic acids and hydrolyzed polyacrylonitrile and their alkali metal salts. A more detailed recitation of superabsorbent polymers is found in U.S. Pat. No. 4,990,541 to Nielsen, the disclosure of which is incorporated herein by reference in its entirety.

Commercially available SAPs include a starch modified superabsorbent polymer available under the tradename SANWET® from Hoechst Celanese Corporation, Portsmouth, Va. SANWET® is a starch grafted polyacrylate sodium salt. Other commercially available SAPs include a superabsorbent derived from polypropenoic acid, available under the tradename DRYTECH® 520 SUPERABSORBENT POLYMER from The Dow Chemical Company, Midland Mich.; AQUA KEEP SA60S manufactured by Seitetsu Kagaku Co., Ltd.; ARASORB manufactured by Arakawa Chemical (U.S.A.) Inc.; Diawet from Mitsubishi, Flosorb made by Floerger, and Aqualic made by Nippon Shokubai (4-1-1, Koraibashi, Chuo-ku, Osaka 541, Phone No. (06) 6223-9111 ,Fax No. (06) 6201-3716)

Further particulate additives may be added to absorbent core 28 in addition to or as a substitute for the foregoing fibrous additives in order to maintain high SAP efficiency. The particulate additives are preferably insoluble, hydrophilic polymers with particle diameters of 100 μm or less. The particulate additives are chosen to impart optimal separation of the SAP particles. Examples of preferred particulate additive materials include, but are not limited to, potato, corn, wheat, and rice starches. Partially cooked or chemically modified (i.e., modifying hydrophobicity, hydrophilicity, softness, and hardness), starches can also be effective. Most preferably, the particulate additives comprise partially cooked corn or wheat starch because in this state, the corn or wheat are rendered larger than uncooked starch and even in the cooked state remain harder than even swollen SAP. In any event, regardless of the particulate additive chosen, one of the important criteria and objectives is to use particulate additives which are hard hydrophilic materials relative to swollen SAP or which are organic or inorganic polymeric materials about 100 microns in diameter. Fibrous and particulate additives can be used together in these absorbent laminates.

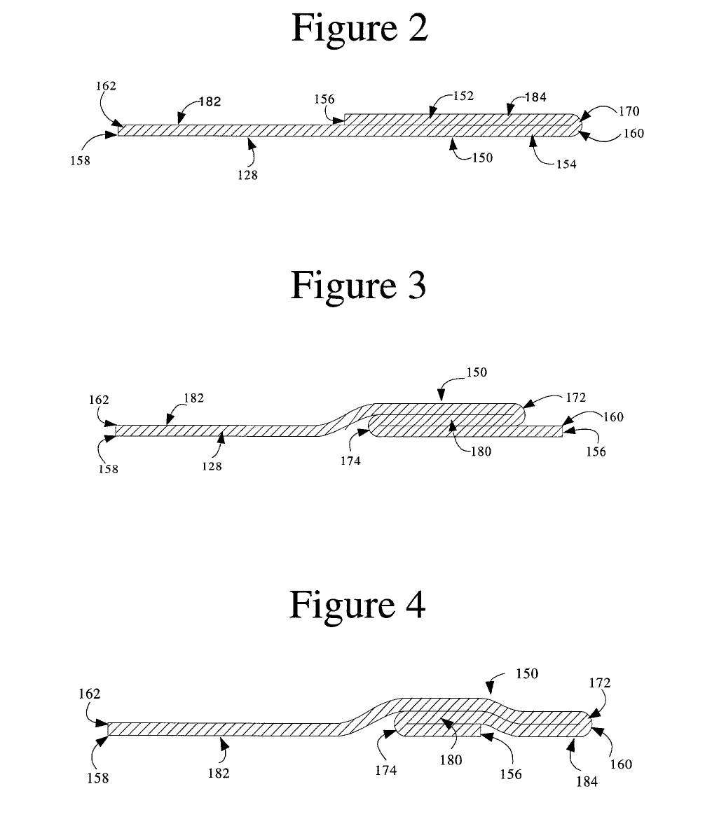

Referring now to FIG. 2, the absorbent core 28 of this invention is initially formed as a length of absorbent core material or absorbent core blank 128 (also referred to herein as core blank). A suitable core blank may be an absorbent laminate having a total basis weight of about 100-400 grams per square meter for example. Roll good core materials known to those of ordinary skill in art are likewise suitable for use as the core blank 128. The core blank 128 is selectively folded in the transverse direction e.g. parallel to lateral axis 102, to create a discreet region or regions with enhanced absorbency potential. FIGS. 2-4 provide exemplary embodiments of absorbent cores 28 with discreet regions of enhanced absorbency potential (“enhanced absorbency”) at a layer region 150 adjacent at least one selectively positioned fold.

FIG. 2 shows a single fold 170 in which a portion of the absorbent core blank 128 is folded such that a first portion 152 is aligned over a second portion 154 of the absorbent core blank 128. The folded first portion 152 is shorter in length than the overall length of the absorbent core 28. Thus, a zone 184 is created at absorbent core end 160 having two layers of absorbent core blank 128 which provides for enhanced absorbency as compared to the region 182 near absorbent core end 162 where there is one layer of absorbent core blank 128. The length of the zone 184 of enhanced absorbency can be adjusted by varying the position of the fold 170.

FIG. 3 shows two folds 172, 174 with both folds of the double fold positioned sufficiently distanced from absorbent core blank ends 156, 158, that a layer region 150 with a zone of three layers 180 of absorbent core material are aligned adjacent to each other at a position inward from the absorbent core ends 160, 162. By adjusting the position of one or both of folds 172, 174, the size of the area of enhanced absorbency and the position of the enhanced absorbency or both may be adjusted. This arrangement permits enhanced absorbency at a position intermediate to the absorbent core ends 160, 162. Further the single layer of absorbent core blank 128 at the absorbent core ends 160, 162 of this embodiment may facilitate reduction of bulk at the more visible areas of the garment adjacent the ends of the absorbent core.

FIG. 4 is an alternative embodiment of an absorbent core with two folds 172, 174. This embodiment provides for three zones 180, 182, 184 having 1, 2 or 3 layers of absorbent core blank 128. As shown the first fold of the two folds (first fold) 172 is positioned coinciding with absorption core end 160 and the second of the two folds (second fold) 174 is positioned some distance inward from the first fold such that the length between first fold 172 and the second fold 174 is greater in length than the distance between the second fold 174 and absorbent core end 156. A zone 184 of the absorbent core near end 160 has two layers of absorbent core material, while an internal zone 180 of the absorbent core 28 has 3 layers of absorbent core blank and a zone 182 near absorbent core end 162 has a single layer of absorbent core blank. By adjusting the positions of the two folds 172, 174 it is possible to adjust either or both of the area and the position of the region of layers or zones of enhanced absorbency. In this exemplary embodiment two zones with different levels of enhanced absorbency over the single layer of absorbent core blank 128 are provided.

The embodiments shown in FIGS. 2-4 are exemplary and skilled artisans are capable of designing other configurations and other positioning of folds using the guidelines provided herein. The absorbent core embodiments shown in FIGS. 2-4 have one or two folds. More folds may be used creating cores with further enhanced absorbance, more zones of different absorbency or both. Factors that should be considered when adding additional folds and positioning folds include absorbency needs and ultimate impact on wearer (user) comfort. The folds may be formed in the manufacturing process in the machine or cross direction (also referred to herein as transverse direction). Given the design of commercially available manufacturing equipment and the typical desired positioning of enhanced absorption, folding in the cross direction is preferred in many embodiments.

Optimum positioning of the folds of the absorbent core may be determined using a Distribution Index calculated according to general formula I, as follows:

$${DI}_{( i , j )} ( \frac{g}{{m}^{2}} ) = \frac{100 ( \frac{cm}{m} )}{N} {\sum }_{T - 1}^{N} \frac{{BW}_{τ}}{( {Dist}_{T} + 7.62 )} \qquad ( I )$$

wherein N is the total number of core cells of the core, each core cell of the core corresponding to each of a plurality of 0.75 inch squares on a predetermined grid;

wherein T is each positive integer from 1 to N, each said positive integer corresponding to each core cell of the core in numerical order;

wherein DIST_{T} is a distance in centimeters (cm) between the center of the core cell corresponding to T and point i,j;

wherein BW_{T} is the basis weight of each core cell, each said core cell corresponding to each value for T; and

wherein i,j is a coordinate representing a point on the core.

This DI or DI profile (a plurality of DI values corresponding to more than one point on the core) then is used as a design criteria for preparing improved cores and thus improved absorbent articles. The DI and DI profile can be determined in accordance with the methods described herein, as well as in accordance with the methods described in United States patent application Ser. No. 09/799,071, entitled “Absorbent Article Having An Ideal Core Distribution and Method of Preparing Same,” the disclosure of which is incorporated by reference herein in its entirety.

The DI for a core (absorbent core) as used herein, is determined according to the following methodology, referred to hereinafter as the Baker Method. First, the core of the absorbent article is divided into a plurality of sample cells, each cell being a discrete section on the absorbent article corresponding to a position on a grid. Then, an identifier, T (a positive integer ranging from 1 to the total number of sample cells, identified as N in general formula I), is assigned to each sample cell in the following manner.

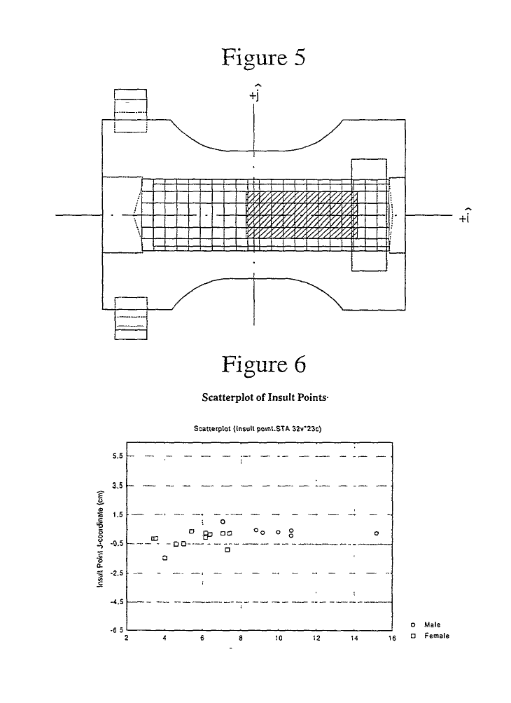

FIG. 5 shows the positioning of the basis weight cells that are numbered 1-N. There are several aspects to the positioning of the cells. Each cell is the same size (in an exemplary embodiment ¾″×¾″). Most cores are not sized so the grid exactly fit the core. The grid is typically positioned such that the front edge of the core exactly matches one edge of the grid so that at least the front edge has full sized cells at its edge. The back of the core will most likely fall such that it will only partially fill the cell. This is also true side to side. The grid is aligned in the j direction such that the i-axis falls exactly on a grid line. This makes cells to the left and right of the i-axis and ensures that there are equal numbers of whole cells on both the left and right side of the core. For those cells that end up only partially filled with core an estimation may be done on the actual area of the core section based on measurements with a ruler or caliper.

In practice the DI can be calculated by theoretical values for basis weight distributions. Calculations based on theoretical core designs yield the DI values for different core designs and provide information for choosing core designs with desired characteristics. For example, this method may be used to determine optimum placement of the folds in a uniform basis weight roll good core in the practice of the present invention.

Referring to FIG. 5, a coordinate system is illustrated that shows the positioning of each of a plurality of points (_{i,j}) on the core of an absorbent article. As illustrated in FIG. 5 positioning of the i coordinate parallels the longitudinal axis 100 of the garment and the j coordinate parallels the lateral axis 102 of the garment.

A Distribution Index may be calculated for any of the points on the core. Thus, the Distribution Index for a certain point on the core is denoted as Distribution Index_{i,j} (DI_{i,j}). As shown in FIG. 5, the values for i indicate a distance (in cm) along the lengthwise direction of the core measured from the lengthwise fold (lengthwise center) of the absorbent article. As shown, at the fold, the value for i is 0. The values for i are positive values from the fold to the front of the article and negative values from the fold to the back of the article. As used herein, the front of the article corresponds to the front of a wearer of the absorbent article and the back of the article corresponds to the back of a wearer of the absorbent article. In FIG. 5, the front is shown on the right-hand side of the absorbent article.

The values for j likewise indicate a distance (in cm) along the width of the core measured from the fold (crosswise center) in the direction of the width of the core. The values for j are positive extending from the left of the centerline of the core and negative extending from the right of the centerline of the core, said left and right corresponding to the left side and the right side of the wearer, respectively. In FIG. 5, the left of the centerline is shown above the centerline. In this manner, the absorbency and other characteristics of various absorbent articles are compared by identifying and comparing the DI at certain points on the cores of absorbent articles. Further, an improved absorbent article can be designed by placing a certain DI at desired points on the core to achieve a desired effect.

Referring to FIG. 5, for the purposes of determining the DI, a sample must be prepared. For example, where the absorbing article is a diaper, the sample is prepared as follows. First, the diaper is opened flat with the topsheet up. Then, the inner leg gathers are carefully removed without making tears in the topsheet. Next, in such a manner as not to disrupt the core or create holes from which SAP can spill, the leg gathers are cut away so that the core of the diaper will lay flat. At the front of the diaper, the width of the diaper core is measured and the center point is determined and marked with a tick mark. This measurement is then repeated at the rear of the diaper. A line then is drawn on the diaper that connects these tick marks. This line is referred to as the centerline. In this manner, a sample is prepared which then is available for subsequent testing.

The DI for a point on a sample of an absorbent article then is tested in the following manner, according to the Baker Method. The front of the sample is placed on a steel rule die grid. The centerline of the sample is aligned with the center blade of the die grid. The center blade then is marked with tick marks and an arrow pointing toward the front direction. Then, the die containing the sample is placed on a USM Platten die press machine such that the sample is facing up. A small stack of about 10 sheets of paper is placed on top of the sample and a plastic cutting board is placed over the sheets of paper. It is preferred to ensure that the plastic cutting board covers all blades of the die. A clean and thorough cut then is made into the sample using the USM Platten die press machine. The cut paper must then be carefully removed without disrupting the topsheets on each of a plurality of sample cells that have been formed by the cut, each said sample cell corresponding to each discrete square section on the grid. Being careful not to spill any of the SAP, the remaining intact sample is set aside for later use. An arrow is drawn on the sample to indicate the forward direction. A 10 inch by 10 inch likeness of the die is drawn on grid paper, leaving ample space for writing 3 lines of measurement for each sample cell. The measurements then are recorded on a paper that has been pre-labeled with the grid coordinates.

Using a knife, a first square of the sample is carefully removed from the die and weighed to the nearest 0.001 gram. This weight then is recorded on the first line in the grid square corresponding to the sample cell. Next, all non-wovens, backsheets and topsheet are removed from the scale. The weight then is measured again and recorded on the second line in the corresponding grid square. If the core portion of the square is smaller than the 0.75 inch by 0.75 inch cut, the average width and average height to the nearest millimeter (which allows calculation of the area of the remaining piece) of the core is measured with electronic calipers (in mm). These measurements then are recorded on the third line in the corresponding grid square. The sample cell that has just been measured then is discarded and each of the remaining sample cells is treated in the same manner as the first data cell. Once the entire die has been measured and recorded, an annotation is made regarding the portion of the absorbent article that was measured on the top of the grid sheet (i.e. front, middle, back). The above procedure then is repeated until the entire core has been cut, weighed and recorded.

The basis weight for each cell then is determined according to general formula II or general formula III. In particular, for all cells where the core completely covers the entire 0.75 inch×0.75 inch square area of the cell, the basis weight is calculated using general formula II, as follows:

$${BW}_{T} ( \frac{g}{{m}^{3}} ) = \frac{{SampleWeight}_{T} ( g ) - {SurroundsWeight}_{T} ( g )}{0.003629 ( {m}^{2} )} \qquad ( II )$$

Alternatively, for all cells where the core does not completely cover the entire 0.75×0.75 inch square area of the cell, the basis weight is calculated using general formula III.

$${BW}_{T} ( \frac{g}{{m}^{2}} ) = \frac{{SampleWeight}_{1} ( g ) - {SurroundsWeight}_{1} ( g )}{width ( mm ) \times length ( mm )} {( \frac{100 ( mm )}{m} )}^{2} \qquad ( III )$$

As used herein, the term “SampleWeight_{I}” refers to the weight of each sample cell. “SurroundsWeight_{T}”, as used herein, refers to the weight of the backsheet, topsheet and other non-wovens that are not part of the core for each sample cell. The weight may be determined by using a scale, a balance or any such means.

Accordingly, in the above manner, the basis weight for each core cell (i.e., each discrete square section of the core corresponding to each sample cell), as designated as BW_{T}, is determined. The basis weight for each core cell BW_{T}, calculated using general formulas (II) or (III), as described above, then is used in general formula (I) for calculating the DI of the core of the absorbent article at a particular point.

The DI is thus finally determined by using the following general formula:

$${DI}_{( i , j )} ( \frac{g}{{m}^{2}} ) = \frac{100 ( \frac{cm}{m} )}{N} {\sum }_{T = 1}^{N} \frac{{BW}_{r}}{( {Dist}_{T} + 7.62 )} \qquad ( I )$$

wherein N is the total number of core cells of the core, each core cell of the core corresponding to each of a plurality of 0.75 inch squares on a predetermined grid;

wherein T is each positive integer from 1 to N, each positive integer corresponding to each core cell of the core in numerical order;

wherein DIST_{T} is a distance in centimeters (cm) between the center of the core cell corresponding to T and point i,j;

wherein BW_{T} is the basis weight of each core cell, each core cell corresponding to each value for T; and

wherein i,j is a coordinate representing a point on the core.

BW_{T} can be determined using any conventional means as would be well known to persons of ordinary skill in the art, using the guidelines provided herein. Accordingly, a DI for any point on the core of an absorbent article may be determined. The DI values obtained in the above manner have a wide range of uses. For example, a graphical representation of the absorbent article's DI values may be developed and compared to the graphical representation of other absorbent articles, without limitation. Further, for example, a DIₘₐₓ, DIₘᵢₙ, DI_{average}, DIₘₐₗₑ, DI_{female}, a DI at any desired point, or other such values may be determined and compared to the corresponding values of other absorbent articles, without limitation.

Cores having a desirable DI profile, DI at a certain point (e.g., at the male or female insult point, without limitation), DIₘₐₓ, DIₘᵢₙ or combination thereof, may be prepared using conventional methods that are readily known and available to persons of ordinary skill in the art, in combination with the technique for determining a Distribution Index (DI) described and claimed herein.

Absorbency distribution in absorbent articles may be compared through a comparison of various DIs. The various DIs include, for example, DIs measured at comparative points i,j on two or more cores, the maximum distribution index (DIₘₐₓ) on the cores, the minimum distribution index (DIₘᵢₙ), the average distribution index (DI_{average}), the distribution index at a point or a plurality of points on the cores, DI profiles or combinations thereof, without limitation. The DI for a plurality of points on a core can form a distribution index profile which is compared to the DI profile corresponding to the same plurality of points on another core.

For example, a higher DI at a certain point on the core, indicates that a greater proportion of absorbent capacity is closer to the insult point (i.e., the point at which fluid enters the core). Accordingly, the higher DI at a certain point or at a plurality of points, relative to the DI at other points, confers advantages in terms of absorbent efficiency, as well as cost efficiency.

A DI profile may be fitted to the gaussian equation of formula IV:

$$Y = {Y}_{o} + {ae}^{[ - \frac{1}{2} {( \frac{x - {x}_{o}}{b} )}^{2} ]} \qquad ( IV )$$

where xₒ is the distance along the midline corresponding to DIₘₐₓ; a is the DI_{average}; b is the standard deviation of the curve; Y is the distribution index at a certain point; x is the distance at a certain point along the centerline; Y₀ is DIₘᵢₙ.

Accordingly, there is an optimal DIₘᵢₙ that provides the maximum amount of cost effectiveness that can be obtained by minimizing absorbency in the back of the diaper while not minimizing the absorbency to so great an amount that the core would not meet minimal performance demands.

In this manner, it is possible to obtain gaussian equations that define a desirable absorbency profile. This equation can be used as a design parameter in determining number of and positioning of folds to form absorbent cores having one or more desired characteristics.

In exemplary embodiments of the invention differences related to male and female insult point positioning may be identified by the DI profile for males (DIₘₐₗₑ) and females (DI_{female}). In this manner, a core may be provided that is optimal for both male and female use (e.g., unisex use), or selectively optimized for either male or female. Further DI profiling may be useful for optimizing absorbency for a particular age group.

For example, the male insult point was determined to be 12 cm from the top of the core, and the female insult point was determined to be 16 cm from the top of the core. The insult points for both males and females will vary according to the age of the baby. As used herein in FIGS. 6, 7 and 8, male, insult points and female insult points are both intended to be age specific terms.

Table V shows various parameters for male and female babies participating in a study to determine the site of male and female insult points.

TABLE V

| | Average | | Standard Deviation | | | |
|---|---|---|---|---|---|---|
| | Male | Female | p= | Male | Female | p= |
| Weight (lbs) | 27.7 | 24.8 | 0.04 | 3.2 | 2.8 | 0.65 |
| Waist (mm) | 461 | 437 | 0.28 | 12 | 55 | 0.00 |
| Thigh left (mm) | 275 | 255 | 0.29 | 28 | 45 | 0.24 |
| Thigh Right (mm) | 273 | 253 | 0.14 | 26 | 32 | 0.63 |
| Hip to Hip (mm) | 185 | 177 | 0.42 | 20 | 25 | 0.62 |
| Hip to ground | 398 | 405 | 056 | 15 | 30 | 0.09 |
| (mm) | | | | | | |
| Initial Tab-Tab | 87 | 76 | 0.20 | 18 | 19 | 0.99 |
| (mm) | | | | | | |
| Used Tab-Tab (mm) | 83 | 73 | 0.12 | 17 | 13 | 0.34 |

FIGS. 6, 7 and 8 show graphs that illustrate the results from the study to determine the site of insult points for male and female babies. In particular, FIG. 6 shows various insult points by gender as a function of the distance from the j-axis (the fold line of the product.) and distance from the centerline along the core.

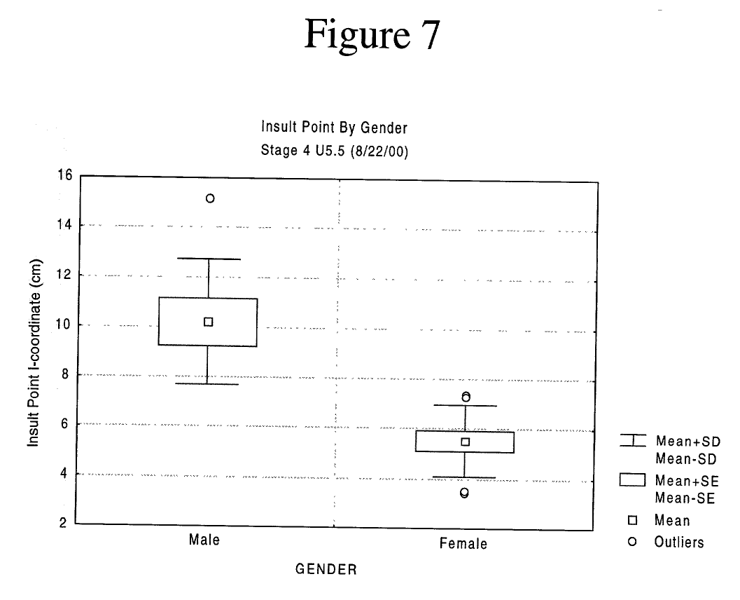

FIG. 6 shows the results of the insult points study for male and female babies as a function of gender and distance from the center of the diaper in centimeters (cm). As shown in the figure, the male insult points are on average about 10.3 cm from the center (where i,j is 0,0) of the diaper and the female insult points are on average about 5.6 cm from the center of the diaper. Moreover, according to the study, male insult points are more variable than female insult points. This is shown in FIG. 7 by the standard deviation for males which equals about 2.5 cm as opposed to the standard deviation for females, which equals about 1.5 cm. As used herein, male insult point refers to average male insult point and female insult point refers to average female insult point. Persons of ordinary skill in the art appreciate that the insult point will vary as a function of age of the child. Accordingly, the present invention contemplates adjustments to tailor the absorbent article to children of different ages, which is well within the skill of the art.

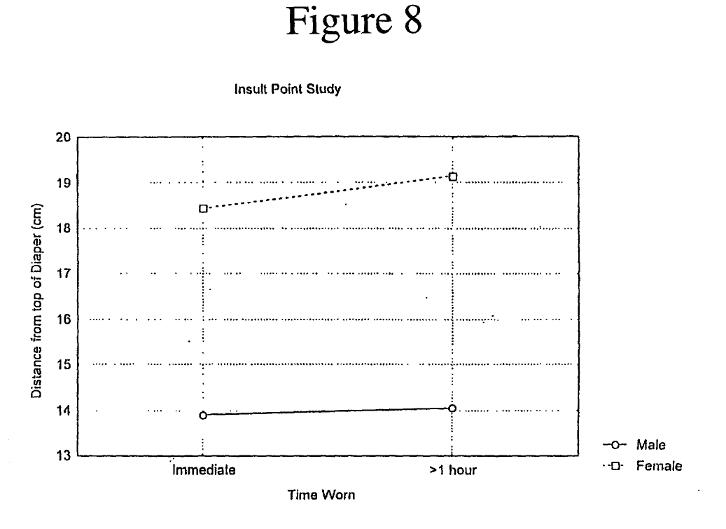

FIG. 7 shows the results of the insult points study for males and females with regard to variations and insult points over time. The graph in FIG. 8 shows the changes in insult point as a function of time and distance from the top of the diaper (cm). As the graph illustrates, over time the insult point appears to move up. This is likely due to sagging of the diaper.

In accordance with an implementation of the invention, changes in position of the insult point caused by sagging can be accounted for by tailoring a Distribution Index Profile of an absorbent article in accordance with the known changes in position of the insult points that occur when the diaper begins sagging. In particular, the DI may be substantially similar in the area around and between the original insult point and the post-sagging insult point. Thus, absorbency is provided in the areas of greatest need during the course of use of the diaper by the wearer. In this manner, an absorbent article having superior longevity may be provided by positioning enhanced absorbency in this region. This would be particularly useful, for example, for an extended-use diaper such as a nighttime diaper (e.g., a diaper intended primarily for use during the nighttime) and/or a travel diaper (e.g., a diaper intended primary for use during travel or a long road trip, or during any somewhat long period of time during which changing the diaper would be generally inconvenient), without limitation.

By applying the Distribution Index Profiling described herein, a Distribution Index (DI) may be determined for a given configuration and basis weight of base material (absorbent core blank). For illustrative purposes we consider the use of a base material having a basis weight of 475 g/m² to form an absorbent core having a finished length of 381 mm in exemplary configurations of the types shown in FIGS. 2, 3 and 4.

For a configuration as shown in FIG. 2 in which the folded zone 184 is 245 mm in length, a female DI of 1025 g/m² and male DI of 1036 g/m² may be achieved using an unfolded linear length of core blank of 68.6 cm.

For a configuration as shown in FIG. 3 in which the folded zone 180 is 152 mm in length and the length from fold 172 to absorbent core end 160 is 102 mm, a female DI of 1239.0 g/m² and a male DI of 1261.1 g/m² may be achieved using an unfolded linear length of core blank of 72.1 cm.

For a configuration as shown in FIG. 4 in which the length from core blank end 156 to fold 174 is 102 mm, and the length from fold 174 to fold 172 is 152 mm, a female DI of 1114.8 g/m² and a male DI of 1152.9 g/m² may be achieved using an unfolded linear length of core blank of 73.7 cm.

As one of ordinary skill in the art will recognize, these three examples are representative of the type of information that may be calculated and utilized in selecting positioning, width, and number of folds to use to create an absorbent core with the desired features. The above examples are provided as illustrative examples and are not intended to limit the scope of the embodiment of the invention. We note that for the examples provided, the Distribution Index values achieved per unfolded linear length of base material used are quite favorable compared to known absorbent cores in commercial use.

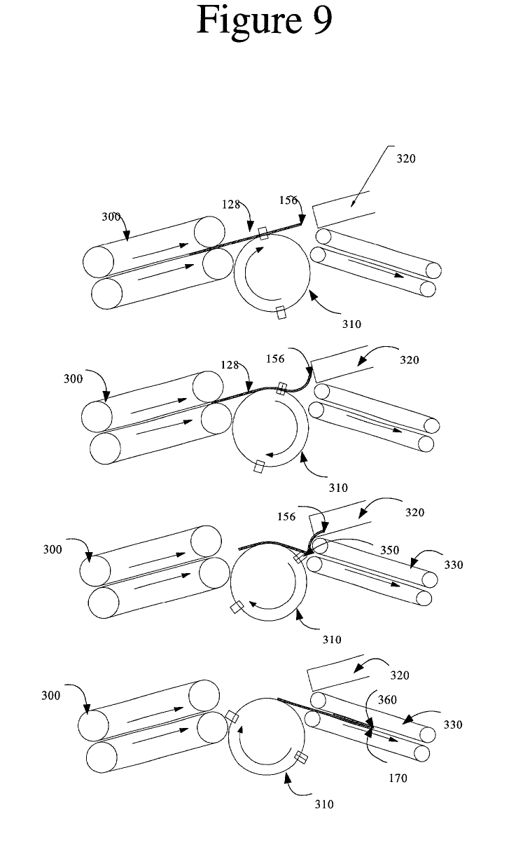

Referring to FIG. 9 an exemplary embodiment of a method for making a single fold in an absorbent core is shown. It should be understood, however, that this is exemplary of one of a number of methods in which folding may be accomplished. As shown in FIG. 9, the length of absorbent core material (absorbent core blank 128) is moved through a set of rollers 300 to a rotating drum 310. As the absorbent core blank 128 passes over the drum 310 the grouping projections 350 group the roll good core blank 128 and hold a portion of the roll good core blank 128 against the drum 310. The leading end 156 of the absorbent core blank 128 is drawn upward by the application of chamber 320, which preferably is a vacuum chamber 320. As the drum 310 turns the absorbent core blank 128 is advanced and, as shown in FIG. 9, a bend 350 is created in the absorbent core at 128 a position inward from the end 156 held by the vacuum. As the drum 310 continues to rotate, the grasping projections 350 release the roll good core blank 128, and the absorbent core blank 128 is forced through a second set of rollers 330 with the bend 350 becoming the leading edge 360. The second set of rollers 330 apply pressure thus creating a single fold 170 of the type shown in FIG. 2.

Those skilled in the art will appreciate that a plurality of folds can be made in absorbent core blank 128 in any particular configuration by repeating the procedure described above with reference to FIG. 9 any number of times. For example, another rotating drum and chamber may be disposed downstream from the second set of rollers 330 to form a second fold.

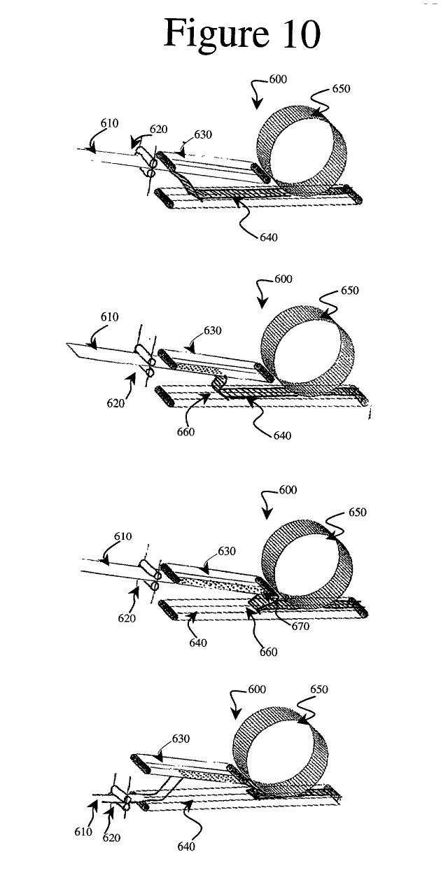

Alternatively, referring to FIG. 10, a continuous web 610 of core material (roll good core) may be fed to a folding apparatus 600. The folding apparatus 600 may make one or more folds in the web, pass a length of web that has no folds and make another one or more folds in the web. The continuous web may subsequently be cut to form individual absorbent cores for absorbent garments. Continuous web as used herein refers to a length of core material substantially longer than the length of core material used for one absorbent core. Preferably it is a length suitable for making a plurality of absorbent cores.

In an exemplary embodiment the folding apparatus 600 has a pair of moveable feed rollers 620, an infeed belt 630, a takeaway belt 640, and a nip drum 650. In this exemplary embodiment the continuous web 610 is fed into the pair of moveable rollers 620. As illustrated in FIG. 10, the folding process may start with the moveable feed rollers 620 in a position approximately level with the infeed belt 630 and the continuous web 610 dropping directly to the takeaway belt 640. As the folding process proceeds continuous web 610 is advanced onto the in-feed belt 630. As the web is moved along by the infeed belt 630 and takeaway belt 640 a first loop 660 is formed in the continuous web 610. As the continuous web 610 is moved still further along by the in-feed belt 630 and takeaway belt 640 a second loop 670 is formed. As the first loop 660 and second loop 670 are forming, the pair of moveable feed rollers 620 move to a position intermediate the infeed belt 630 and takeaway belt 640 or a position approximately level with the takeaway belt 640. The first loop 660 and second loop 670 are advanced by the infeed belt 630 and takeaway belt 640 to the nip drum 650 where they are pressed together to create two folds and a region of the continuous web 610 which has three layers of core material (e.g., layered region). The continuous web 610 continues to feed along takeaway belt 640 until the pair of moveable rollers 620 return to the start position approximately level with the infeed belt 630 and the folding process begins for another section of the continuous web 610.

In a preferred embodiment the belts 630, 640 are vacuum belts. The vacuum belts 630, 640 of the invention have a series of holes in the belt 630, 640 and a vacuum box under the belt 630, 640 so that materials in contact with the holes will tend to be held in place because of the vacuum. The holes are selectively positioned in the belt 630, 640 and the position of the holes in combination with the length of the belt 630, 640 serve to position the loops 660 and 670. The vacuum belts 630, 640 may be timed and offset specifically to positively control the location of the continuous web 610 during the process. The movable feed rollers 620 are used to bring the roll good core 610 in contact with the vacuum belts 630, 640. The movable feed rollers 620 move up and down to alternately attach the roll good core 610 to the infeed 630 and take away belt 640. The vacuum holes in the take away belt 640 are timed along with the holes in the infeed belt 630 to take control of the formation of the advancing loop (first loop) 666 of roll good core material 610. Additionally the infeed 630 belt in a preferred embodiment may move faster than the take away belt 640. The relative speed of the vacuum belts 630, 640 (and nip drum), the position of the vacuum holes in the belts 630, 640, length of the belts 630, 640 and the timing of the feed rollers are design parameters that define how this system functions and hence on positioning, length of the layers.

The timing of the return of the feed rollers 620 to the start position in combination with the speed at which the roll core web is advanced determines the length of roll good core between layered regions.

As shown in FIG. 10 two loops are formed creating 3 layers of web material in the folded region. This is one exemplary embodiment for provided illustrative purposes . Artisans of ordinary skill are capable of designing other configurations for producing a plurality of loops. Further one of ordinary skill in the art may without undue experimentation create a variety of configurations of folds, positioning of folds, fold lengths and the like based on the disclosure herein.

The invention has been described in connection with the preferred embodiments, these embodiments, however, are merely for example and the invention is not restricted thereto. It will be understood by those skilled in the art that other variations and modifications can easily be made within the scope of the invention as defined by the appended claims.

What is claimed is:

1. An absorbent article comprising:
a liquid permeable top sheet;
a liquid impermeable back sheet associated with said top sheet; and
an absorbent core disposed between the top sheet and the back sheet, the absorbent core comprising an absorbent laminate, the absorbent laminate comprising:
an upper layer near the top sheet;
a lower layer near the back sheet; and
an absorbent layer positioned between the upper and lower layers,
said upper and lower layer comprise a material selected from the group comprise a material selected from the group consisting essentially of tissue, air laid fluff pulp and synthetic non-woven;
said absorbent layer comprising about 10-90% superabsorbent polymer (SAP) by weight; and wherein said absorbent laminate has;
a first end; a second end; at least one transverse fold; and a layer region of two or more layers positioned between said first end and said second end and wherein said layer region has a plurality of layer edges at least one of said plurality of layer edges coinciding with said at least one transverse fold and at least one of said plurality of layer edges positioned at a distance intermediate said first end and said second end.

2. The absorbent article of claim 1, said absorbent layer further comprises fibers selected from the group consisting essentially of tow fibers, cellulose acetate fibers, rayon fiber, LYOCELL fibers, polyacrylonitrite fibers, cotton fibers and cotton linter fibers.

3. The absorbent article of claim 1, said absorbent layer further comprises fibers selected from the groups consisting essentially of surface modified (hydrophilic) polyester fibers and surface modified polyolefin/polyester bicomponent fibers.

4. The absorbent article of claim 1, said absorbent layer comprising about 20-90% SAP by weight.

5. The absorbent article of claim 1, wherein the absorbent layer further comprises particulate additives.

6. The absorbent article of claim 5, said particle additives comprising insoluble, hydrophilic polymers with particle diameters of 100 μm or less.

7. The absorbent article of claim 1, said at least one transverse fold comprises two folds.

8. The absorbent article of claim 1, wherein all of said plurality of layer edges of said layer region are at a position intermediate said first and second ends.

9. The absorbent article of claim 1, wherein the layer region is positioned for a female user.

10. The absorbent article of claim 1, wherein the layer region is positioned for a male user.

11. The absorbent article of claim 1, wherein the article is an extended use diaper.

12. The absorbent article of claim 1, the absorbent layer further comprises a tow fiber material selected from the group consisting essentially of cellulose acetate, cellulose propionate, cellulose butyrate, cellulose caproate, cellulose caprylate, cellulose stearate, highly acetylated derivatives thereof such as cellulose diacetate, cellulose triacetate and cellulose tricaproate, and mixtures thereof.

13. An absorbent core for absorbent garments comprising:
a roll good core portion, said roll good core portion having:
a first end;
a second end;
at least one transverse fold; and
a layer region of two or more layers positioned between said first end and said second end, and wherein said layers have a plurality of layer edges, at least one of said plurality of layer edges coinciding with said at least one transverse fold and at least one of said plurality of layer edges positioned at a position intermediate said first end and said second end.

14. The absorbent core of claim 13, said roll good core comprising fibrous material selected from the group consisting essentially of tow fibers, cellulose acetate fibers, rayon fibers, LYOCELL fibers, polyacrylonitrile fibers, surface modifier (hydrophilic) polyester fibers, surface-modified polyolefin/polyester bicomponent fibers, surface-modified polyester/polyester bicomponent fibers and cotton fibers.

15. The absorbent core of claim 13, said roll good core portion further comprising about 10-90% SAP by weight.

16. The absorbent core of claim 13, said roll good core portion further comprising about 20-90% by weight SAP.

17. The absorbent core of claim 16, said roll good core portion further comprising particulate additives.

18. The absorbent core of claim 13, wherein all of said plurality of layer edges of said layer region are positioned intermediate said first and second ends.

19. The absorbent core of claim 13, wherein the at least one transverse fold comprises two folds.

20. The absorbent core of claim 13, wherein the layer region is positioned for a female user.

21. The absorbent core of claim 13, wherein the layer region is positioned for a male user.

22. A method of manufacturing absorbent core for absorbent garments comprising:
providing a roll good core portion, having a first end and a second end to a folding station including a rotating drum having grouping portions, the first end being the leading end and the second end being the trailing end;
drawing the leading end into a chamber disposed adjacent the folding station;
engaging the roll good core portion with the grasping projections of the rotating drum;
advancing the trailing portions of the roll good core portion along the rotating drum and past the chamber;
creating a fold in the roll good core portion as the trailing portions of the roll good core portion advanced past the chamber;
releasing the leading end from the chamber prior to the trailing end advancing past the chamber on the rotating drum; and
releasing the roll good core portion from the grouping projections.

23. The method of manufacturing absorbent core of claim 22, wherein the method is repeated at least once to provide an absorbent core having a plurability of folds.

24. The method of manufacturing absorbent core of claim 22, wherein the roll good core portion comprises fibers selected from the group consisting essentially of tow fibers, cellulose acetate fibers, rayon fibers, LYOCELL fibers, polyacrylonitrile fibers, cotton fibers, cotton linter fibers, surface modified (hydrophilic) polyester fibers and surface modified polyolefin/polyester bicomponent fibers.

25. The method of manufacturing absorbent core of claim 22, the roll good core portion further comprises a tow fiber material selected from the group consisting essentially of cellulose acetate, cellulose propionate, cellulose butyrate, cellulose caproate, cellulose caprylate, cellulose stearate, highly acetylated derivatives thereof such as cellulose diacetate, cellulose triacetate and cellulose tricaproate, and mixtures thereof.

26. The method of manufacturing of absorbent core of claim 22, the roll good core portion further comprising about 20-90% SAP by weight.

27. The method of manufacturing absorbent core of claim 26, said roll good core portion further comprising particulate additives.

28. The method of manufacturing absorbent core of claim 27, said particulate additives comprising insoluble, hydrophilic polymers with particle diameters of 100 um or less.

29. A method of making an absorbent garment comprising:
supplying a top sheet material;
supplying a back sheet material;
preparing an absorbent core; and
disposing the absorbent core between the top sheet material and the back sheet material,
whereby the absorbent core is prepared by
providing a roll good core portion, having a first end and a second end to a folding station including a rotating drum, the first end being the leading end and the second end being the trailing end;
drawing the leading end into a chamber disposed adjacent the folding station;
advancing the trailing portions of the roll good core portion along the rotating drum and past the chamber;
creating a fold in the roll good core portion as the trailing portions of the roll good core portion advanced past the chamber; and
releasing the leading end from the chamber prior to the trailing end advancing past the chamber on the rotating drum.

30. The method of making an absorbent garment of claim 29, wherein the fold of the roll good core portion is position for male user.

31. The method of making an absorbent garment of claim 30, wherein the fold of the roll good core portion is positioned for female user.

32. A method of manufacturing absorbent core for absorbent garments comprising:
providing a roll good core web having a first web portion, a layer segment and a second web portion;
advancing the roll good core web to a folding station having a first moving belt, a second moving belt and a pressor wherein the first web portion of the roll good core is drawn along the first moving belt and the layer segment portion is drawn along the second moving belt;
creating at least one loop in the roll good core web as the roll good core web is drawn along by the first and the second moving belts;
pressing at least one loop into at least one fold with the pressor;
feeding a second web portion of roll good core web along one of the first and second moving belts; and
cutting a length of roll good core web wherein the length of roll good core web comprises at least one fold and a portion of at least one of the first and second web portions.

33. The method of manufacturing absorbent core of claim 32, wherein the roll good core web comprises fibers selected from the group consisting essentially of tow fibers, cellulose acetate fibers, rayon fibers, LYOCELL fibers, polyacrylonitrile fibers, cotton fibers, cotton linter fibers, surface modified (hydrophilic) polyester fibers and surface modified polyolefin/polyester bicomponent fibers.

34. The method of manufacturing absorbent core of claim 32, creating two loops and two folds.

35. The method of manufacturing absorbent core of claim 22, the roll good core web further comprises a tow fiber material selected from the group consisting essentially of cellulose acetate, cellulose propionate, cellulose butyrate, cellulose caproate, cellulose caprylate, cellulose stearate, highly acetylated derivatives thereof such as cellulose diacetate, cellulose triacetate and cellulose tricaproate, and mixtures thereof.

36. The method of manufacturing of absorbent core of claim 22, the roll good core portion further comprising about 20-90% SAP by weight.

37. The method of manufacturing absorbent core of claim 26, said roll good core portion further comprising particulate additives.

38. The method of manufacturing absorbent core of claim 27, said particulate additives comprising insoluble, hydrophilic polymers with particle diameters of 100 um or less.

39. The method of manufacturing absorbent core of claim 32, the pressor comprising a rotating drum.

40. A method of making an absorbent garment comprising:
supplying a top sheet material;
supplying a back sheet material;
preparing an absorbent core; and
disposing the absorbent core between the top sheet material and the back sheet material,
whereby the absorbent core is prepared by
providing a roll good core web having a first web portion, a layer segment and a second web portion;
advancing the roll good core web to a folding station having a first moving belt, a second moving belt and a pressor wherein the first web portion of the roll good core is drawn along the first moving belt and the layer segment portion is drawn along the second moving belt;
creating at least one loop in the roll good core web as the roll good core web is drawn along by the first and the second moving belts;
pressing at least one loop into at least one fold with the pressor;
feeding a second web portion of roll good core web along one of the first and second moving belts; and
cutting a length of roll good core web wherein the length of roll good core web comprising at least one fold and a portion of at least one of the first and second web portions.

41. The method of making an absorbent garment of claim 40, wherein the fold of the roll good core web is positioned for male user.

42. The method of making an absorbent garment of claim 40, wherein the fold of the roll good core web is position for a female user.
